(19) United States
(12) Patent Application Publication
(10) Pub. No.: US 2011/0129893 A1
(43) Pub. Date: Jun. 2, 2011

(54) MONITORING TARGET ENDOGENOUS SPECIES

(75) Inventors: Maryanne Dalton, Dublin (IE); John Lowry, Dublin (IE)

(73) Assignee: NATIONAL UNIVERSTIY OF IRELAND, MAYNOOTH, County Kildare (IE)

(21) Appl. No.: 12/739,818

(22) PCT Filed: Oct. 21, 2008
(86) PCT No.: PCT/EP2008/064226

§ 371 Date: Jul. 2, 2010

(30) Foreign Application Priority Data
Oct. 24, 2007 (IE) S2007/0774

(51) Int. Cl. C12N 11/02

(52) U.S. Cl. 435/177

(57) ABSTRACT

An electrode comprising: a conducting substrate for detecting an electroactive agent; a catalytic agent held on the substrate for converting a non-electroactive agent to an electroactive agent by a catalytic process which consumes oxygen; and an oxygen reservoir, for releasing oxygen to feed the catalytic process, held within a polymer matrix on the substrate. The electrode allows for detection of materials such as glucose when using oxidase enzymes. The invention solves the problem of fluctuation or depletion of oxygen in the environment in which the measurements are being taken.

### FIELD OF THE INVENTION

The invention is a device, such as a biosensor, than can selectively monitor one of a number of chemicals present in the body for example in the brain. The invention in particular relates to oxidase enzyme-based devices. Such a device, when in the presence of oxygen (O₂), generally liberates electroactive species such as peroxides, for example H₂O₂. Detection of target species can be subject to interference by (other) interfering species. Accordingly the invention is directed toward selectively detecting target species. This is achieved without any substantial interference from such potential interfering species. An electrode can be employed to detect the target species (which in general are liberated electroactive species).

### BACKGROUND AND BRIEF DESCRIPTION OF RELATED ART

The device of the present invention will generally be an electrode-based sensor (“EBS”). The EBS is suitable for use as a biosensor. It may be configured to detect electroactive species which are generated in sufficiently close proximity to the electrode to be detected by the electrode.

Such EBSs are known.

In a biosensor, the target molecule that interacts with the biological sensing element is, in general, non-electroactive. Thus, the biological sensing element needs to be positioned in close proximity to the charged surface to convert the target species into a readily detectable electroactive molecule. A number of biological sensing elements can be employed to do this, but the most commonly employed are enzymes.

The invention in particular relates to oxidase enzyme-based biosensors, which in the presence of O₂ liberate electroactive H₂O₂. Monitoring the levels of H₂O₂ produced allows observation of minute changes in the target molecule concentrations at a real-time level. Thus, incorporation of glucose oxidase (GOx), lactate oxidase (LOx) or glutamate oxidase (GluOx) into the biosensor allows measurement of real-time fluctuations in glucose, lactate, or glutamate concentrations respectively. Real-time in-vivo neurochemical monitoring of these molecules has huge potential in determining and finding possible cures for a number of neuro-degenerative and psychiatric disorders.

Oxidase-based biosensors work by electrochemically detecting H₂O₂ produced through the enzyme-catalysed oxidation reaction with their corresponding target substrate. The main basis for the GOx dominance in this field is because of the importance of glucose monitoring. For example glucose monitoring is important in relation to the disease diabetes mellitus. Glucose determination in various body fluids, such as blood, plasma and urine, remains one of the most common analyses performed in clinical laboratories.

Other contributing factors for neurochemical monitoring includes a requirement for a biosensor with a robust nature, including for example choosing the enzyme for its ability to function under relatively hostile and non-controllable conditions for example the conditions within the body such as within the living brain.

Nevertheless, the necessity of real-time monitoring of other chemical species such as lactate and glutamate is foremost in sensor development. Since neither species is electrochemically active, lactate oxidase (LOx) and glutamate oxidase (GluOx) are required to convert the target species to a detectable form for example by producing electroactive peroxide, for example H₂O₂.

Until now, producing a sensor that can incorporate suitable enzymes, for example incorporating the enzyme into a surface matrix, without hampering enzyme activity or compromising other aspects of sensor behaviour has had little success.

Three factors have limited the successful development of biosensor technology for use in the body such as for in particular the brain. Firstly, creating a biosensor surface environment where the required enzymes can function to their full ability has proven incredibly difficult.

Secondly, in the specific case of biosensors incorporating oxidase enzymes, the catalytic cycle of the enzyme requires a plentiful supply of oxygen whereas the sensors are usually employed in an environment where the endogenous oxygen levels are low and fluctuate (such as the brain). It is necessary to ensure that inadequate oxygen levels, (that is oxygen levels that substantially affect the catalytic reaction resulting in substantially inaccurate measurements) for example inadequate oxygen levels due to fluctuation and/or depletion of oxygen do not affect the measurements taken.

Thirdly, a number of electroactive molecules in the brain can cause significant interference to the signal obtained from the sensor resulting in inaccurate readings/results. A number of easily oxidisable components within fluids within the body can interfere with detection. Examples include naturally occurring substances such as ascorbic acid and uric acid, and medicaments such as acetaminophen (paracetemol).

Most reported biosensors include glucose oxidase-based electrodes. While glucose monitoring is of significant importance, the main reasons for such focus being on the glucose oxidase based sensors are that the enzyme is considered a model enzyme being relatively cheap, highly active, and is much more robust than all other oxidase enzymes. This fact means that there are a wide variety of construction methods which can be used for glucose oxidase based sensors. Such constructions are not generally suitable for use with the relatively less sensitive (lower activity), and fragile enzymes such as LOx and GluOx. The latter enzymes are rarer than glucose oxidase and are more expensive due to their relatively lower stability.

UK patent application no. GB 2 296 773 discloses a polyion complex membrane of an immobilised enzyme formed on an electrode. Examples of polyanions include poly(styrene sulfonic acid). Lactic acid is determined using lactate oxidase as the enzyme. The document mentions using perfluorinated ionomer such as that sold under the trade name Nafion® by DuPont Company as part of a molecular sieve arranged to exclude unwanted interfering substances. The document does not deal with the issue of inadequate oxygen levels.

Systems for monitoring lactate concentrations utilising polystyrene as an immobilisation matrix for lactate oxidase are described by Bolger et al. Real-time Monitoring of Brain Extracellular Lactate (Bolger, F. B., Serra, P. A., Dalton, M., O'Neill, R. D., Fillenz, M. and Lowry, J. P. (2006) Real-time monitoring of brain extracellular lactate. In Monitoring Molecules in Neuroscience, Di Chiara, G., Carboni, E., Valentini, V., Acquas, E., Bassareo, V. and Cadoni, C. (Eds.) University of Cagliari Press, Cagliara, Italy; pp. 286-288). Dalton et al. “A polystyrene-based glucose biosensor for in vivo neurochemical analysis” describe a system for eliminating ascorbic acid interference in a biosensor (Dalton, M. and Lowry, J. P. (2001) A polystyrene-based glucose biosensor for in vivo neurochemical analysis. In Monitoring Molecules in Neuroscience, O'Connor, W. T., Lowry, J. P., O'Connor, J. J. and O'Neill, R. D. (Eds.) University Press, University College Dublin, Ireland; pp. 49-50) a platinum electrode is coated utilising poly(O)phenylenediamine (PPD) to bind Gox. Dalton et al. “Utilisation of polystyrene as an immobilisation matrix in the construction of a lactate biosensor for real-time in vivo monitoring” (Dalton, M. and Lowry, J. P. (2003) Utilisation of polystyrene as an immobilisation matrix in the construction of a lactate biosensor for real-time in vivo monitoring). In Monitoring Molecules in Neuroscience, Kehr, J., Fuxe, K., Ungerstedt, U. and Svensson, T. H. (Eds.) Karolinska University Press, Stockholm, Sweden (ISBN: 91-7349-589-1); pp. 437-439) describe a lactate sensor which employs polystyrene to form an immobilisation matrix for a desired enzyme.

Shengtian Pan and Mark A. Arnold Talanta, 43 (1996) 1157-1162 describe adding a thin layer of the DuPont product Nafion® between the electrode surface and the immobilised layer of glutamate oxidase to enhance selectivity. The layer is employed on the basis that the Nafion® layer electrostatically repels anions, such as ascorbate while freely passing hydrogen peroxide. The authors of this paper also report a reduction in the electrode response to hydrogen peroxide.

Where prior art does describe methods of construction of non-glucose oxidase-based sensors, such as LOx and GluOx based biosensors, there are a number of limitations which have not been considered which make them unsuitable for use in-vivo. The most significant issues which have not been addressed are that: (i) it is assumed that the vital O₂ mediator will exist in the target environment at levels high enough to ensure that it does not interfere with stability and sensitivity of the signal (—this is a false assumption in the case of a sensor for use in the body in particular the brain); (ii) Interference rejection issues have not been investigated fully, particularly in vivo; (iii) the biocompatibility of the sensor surface with the target environment has not been considered (experiments conducted utilising artificial solutions in vitro) (iv) the sensors are not generally constructed in a manner easily reproducible on a commercial scale or with amounts or kinds of materials which are economically viable.

In an attempt to deal with oxygen deficiency Wang et al. in J. Wang, L. Chen and M. P. Chatrathi, Analytica Chimica Acta, 2000, 411, 187 and in J. Wang, F. Lu, J. Am. Chem. Soc. 1998, 120, 1048-1050 have prepared fluorochemical carbon-paste needle-shaped electrodes which were employed to detect glucose levels in solutions which had been purged of oxygen utilising helium. The electrodes are said to be oxygen insensitive. Nafion® by DuPont and Kel-F (polychlorotrifluoroethylene oil binder) by 3M are mentioned as suitable fluorochemical carbon-paste materials.

Solutions to the oxygen-deficiency problem such as those suggested by Wang with carbon paste electrodes create problems of their own. For example, where the technology has attempted to correct the problems arising from low environmental O₂ mediator concentration, the incorporated metal particle substrate can leach from the sensor surface. The problem then becomes two-fold; firstly, the metal substrate cannot function to enable enzymatically generated H₂O₂ oxidation to occur and secondly, the leached substrate can upset the endogenous environment causing unrealistic situations at the sensor surface. Another example, with similar effects, would be that the sensor surface is incompatible, biologically, with the target environment. This lack of biocompatibility can result in imposed changes in the endogenous behaviour around the sensor surface, leading to unrealistic chemical information. One example of this would be removal of the pasting oil (for example Kel-F) from the carbon paste surface by lipids in the brain tissue.

Carbon-paste electrodes vary based on amount, type and the packing of the paste, which is variable, even where generally consistent techniques are employed to make the electrodes. Their method of construction is labour intensive and the fact that they are modified by brain tissue leads to limitations with respect to their commercial production.

Notwithstanding the various teachings of the prior art, there is still a necessity to provide a reliable and accurate EBS which can accurately sense the target species, reject interfering species, and operate in circumstances were there may be depleted oxygen levels.

### SUMMARY OF THE INVENTION

The present invention provides an electrode comprising:
(i) a conducting substrate for detecting an electroactive agent;
(ii) a catalytic agent held on the substrate for converting a non-electroactive agent to an electroactive agent by a catalytic process which consumes oxygen;
(iii) an oxygen reservoir, for releasing oxygen to feed the catalytic process, held within a polymer matrix on the substrate.

An electrode according to the present invention is capable of providing a signal directly proportional to the concentration of the specific target substrate. The present invention ensures that a steady oxygen supply is available to the enzyme in order to ensure accurate monitoring of the target substrate—that is inadequate oxygen levels which substantially affect measurements are no longer a problem. In the present invention the process is generally an oxidative catalytic process, which involves oxygen. The electrode of the invention is capable of operating within oxygen-depleted environments—that is where the levels of oxygen are not sufficient to renew the catalyst for further catalytic action (affecting sensitivity and thus detection of the electroactive agent being only of the amount converted rather than the true amount). Provided the oxygen reservoir can maintain an oxygen concentration in-line with physiological oxygen concentration (30-50 μM) or above, the biosensor signal should be reliable and reproducible.

The conducting substrate can be constructed on any surface that can be charged with an electric voltage. Conducting substrates suitable for use within the present invention include non-metallic conductors such as carbon fibres and metallic conductors including those constructed of noble metals such as Pt, Ag, Au, Ru, Rh, Pd, Re, Os, Ir and combinations thereof. Substrates of the present invention will be self-supporting, that is they do not rely on any other material for support (for example to hold them together). Their structural integrity is sufficient. In contrast to pastes and other non-self-supporting materials the substrates of the present invention will not flow like a liquid under stress forces. Included within the present invention are conducting substrates formed by a conducting material coated onto a non-conductive material.

Typically the conducting substrate of the present invention will take the form of a length of conducting material such as a wire e.g. Pt or carbon fibre.

For applications with the body it is desired that the electrode be minimally invasive. For that reason it is desirable that the electrode has dimensions no greater than 100 mm in length, 0.5 mm in cross-sectional width and no less than 10 mm in length. Typical dimensions range from 10 mm to 100 mm.

Suitable catalytic agents include: enzymes, proteins, antibodies, nucleic acids and receptors. Of particular interest within the field of neurological applications include oxidase materials, particularly those capable of oxidising at least one of glucose, lactate and glutamate. Examples include glucose oxidase, lactate oxidase and glutamate oxidase.

The catalytic process is oxygen dependent. The catalytic cycle will include molecular oxygen. The molecular oxygen is taken from the environment surrounding the electrode and from the oxygen reservoir. The oxygen is involved in the creation of the electroactive species with concomitant regeneration of the enzyme catalyst. Suitably the electroactive agent is hydrogen peroxide. A typical catalytic cycle is as follows:

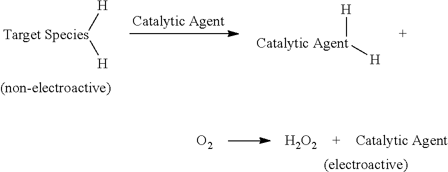

The oxygen reservoir desirably comprises a polymeric material which is typically oligomeric in nature, having for example less than five monomer units. The oxygen reservoir releasably (reversibly) retains oxygen which releases to keep the catalytic agent regenerated and typically compensates for any shortfall in oxygen otherwise available from the environment. Suitable materials include halogenated materials such as fluorinated or chlorinated materials in particular, perfluoro, perchloro and perfluorochloro polymers. In contrast to prior art scenarios where fluoro or chloro polymers have been employed as a permaselective barrier within electrode constructions to prevent interfering electroactive materials from being detected the present inventors have surprisingly found that an oxygen reservoir can be within a polymeric matrix without loss of the oxygen reservoir functionality.

One desired construction is where the oxygen reservoir material is dispersed within a polymeric layer, optionally within discrete areas of the polymeric layer.

The amount of oxygen reservoir material typically employed will be from 0.1-30% by weight based on the weight volume ratio of the material to its dissolution agent. Typically the ratio of oxygen reservoir material to the polymer layer in which it is supported is 1:50 or less such as 1:30 or less for example 1:20 or less such as about 1:10. The polymer matrix thus contains oxygen reservoirs that enable a catalytic agent such as an enzyme to function independently of the fluctuating oxygen levels in the target environment, which will automatically refill from local oxygen level ensuring a constant oxygen supply to the enzyme. Sensors which are based on electrodes of the invention have been fully developed and characterised to determine sensitivity, selectivity and stability. The biosensors have been successfully used in the target “in-vivo” brain environment to monitor levels of glucose, lactate and glutamate.

The use of an oxygen reservoir of the type described above deals with the oxygen depletion aspect of the invention.

A further aspect of the invention includes providing a suitable permselective barrier on the conducting substrate for excluding from detection non-targeted species, and more potentially interfering species such as those described above. This is particularly useful for in vivo measurements. It will be appreciated that the barrier can be chosen to exclude undesired interfering non-target species. In non-in vivo scenarios where there is not any interfering species the permselective barrier need not be employed. Any suitable barrier may be employed but in the present invention it is desirable to utilise a polymeric barrier. For example a layer of polymeric material on the conductive substrate can be employed. Suitable materials include non-conductive polymers. One suitable material is PPD. Others include polyphenols, polycarbonates and poly(acrylic acids). Optionally the permselective barrier is electrochemically grown. Desirably the barrier is substantially uniform across the conductive substrate. Typical permaselective barriers are 1 to 500 μm thick, such as from about 2 to 400 μm thick, for example 3 to 200 μm thick, more desirably 10 to 100 μm thick.

Within the present invention it is desirable to employ a polymeric matrix which immobilizes the catalytic agent but which does not interfere with the detection of target electroactive species by the conductive substrate. Any suitable polymeric matrix may be employed. It is desirable to use a non-electrochemical method of application of the matrix material. Examples include dip-coating and droplet evaporation and combinations thereof. Dip coating is a particularly convenient technique. Of particular interest within the present invention are polymers formed from curable (monomeric) materials for example those that are liquid. Liquid materials such as those just mentioned can be applied by dip-coating. One such suitable polymer is polystyrene which can be applied as the monomer styrene and later cured. In simple terms the matrix acts as a binding material as it holds other species onto the electrode while allowing free movement of potential analytes through the matrix for detection at the conducting substrate.

The matrix desirably has the following characteristics: ability to immobilise the catalytic agent in a stable and active form, ability to allow unhindered access of substrate molecules to the catalytic agent and resultant signal generating products to the electrode surface, be unreactive with other chemical agents such as stabilisers, and be biocompatible. Desirably the matrix overlies a permselective material. The matrix will generally have a thickness of 0.01 mm to 0.5 mm such as 0.03 to 0.4 mm, suitably 0.05 to 0.3 mm for example about 0.1 mm.

It is desirable that the oxygen reservoir material is incorporated into the same matrix as the catalytic agent. Where a polymeric matrix is formed it is thus desirable to incorporate both the catalytic agent and the oxygen reservoir material into the matrix. This may be done by introducing both components into a curable material and later curing the material. Dip-coating can be employed to add each of the matrix material, the catalytic agent and the oxygen reservoir material. It is to be noted that the oxygen reservoir material is desirably already in its polymeric form. In such a case it may be appropriate to utilise a solution of the oxygen reservoir material for application thereof.

In one desirable arrangement, curable material to form the matrix and the oxygen reservoir material are mixed in a desired ratio prior to application thereof to the electrode. Suitable ratios include those in the range from 100:1 to 1:1, more particularly those in the range from 40:1 to 10:1.

Within the present invention it is also useful to include a cross-linking agent, in particular one that can more securely bind the catalytic agent into the matrix. Such a material can be utilised in amounts from 0.1-50% based on the weight of the agent with respect to its dissolution material (e.g. lower aliphatic alcohol). Suitable materials include dialdehydes e.g. gluteraldehyde, diisocyantes e.g. diisocyanato alkyls and aromatics, 1,4-diisocyanatobutane, and diepoxides e.g. 1,2,7,8-diepoxyoctane and 1,2,9,10-diepoxydecane.

Where appropriate it is desirable to include within the matrix a stabiliser, which acts to stabilise the catalytic agent. This applies particularly where the catalytic agent is a biological catalyst such as an enzyme. The stabiliser may be employed to ameliorate any potential for denaturing. Suitable materials include, serum albumins such as bovine serum albumin, N-γ-Acetyldiaminobutyrate, Trehalose, glycerol and dithiothreitol and combinations thereof. Typically such stabilisers are employed in amounts from about 1 to about 20% by weight. Such stabilisers can inhibit denaturation, improve cross-linking properties and provide additional mechanical strength.

Additional protective agents which may be employed include polyethyleneimine, NaCl, sorbitol, polypropyleneimine, poly(N-vinylimidazole), polyallylamine, polyvinylpyridine, polyvinylpyrollidone, polylysine, protamine and combinations thereof. Typically such components are employed in amounts from 1 to 10% by weight.

A matrix comprising some or all of the components mentioned above is hospitable to a range of oxidase enzymes and is distinct from prior art where the immobilisation matrix is primarily designed around the behaviour of an individual enzyme. The electrodes of the invention have demonstrated versatility in end-use to a range of catalytic agents.

The invention also extends to a method of constructing an electrode comprising:
(i) providing a conducting substrate for detecting an electroactive agent;
(ii) providing on the substrate a catalytic agent for converting a non-electroactive agent to an electroactive agent by a catalytic process which consumes oxygen;
(iii) providing within a polymer matrix on the substrate, an oxygen reservoir, for releasing, in use, oxygen to feed the catalytic process.

Such an electrode may be additionally be provided with the additional features/be constructed as set out above.

The invention extends to an electrode and a method substantially as described herein with reference to the examples.

### BRIEF DESCRIPTION OF THE DRAWINGS

FIG. 1 is a schematic representation of an electrode of the present invention.

FIG. 2 is a plot of current (nA) versus time depicting the stability in sensitivity to glutamate of the biosensors in fluctuating oxygen conditions, where oxygen levels are monitored using an independent oxygen biosensor.

FIG. 3 is a plot of current (nA) versus time depicting dependence of biosensors on oxygen by displaying the changing sensitivity of the sensors in fluctuating oxygen conditions in the absence of an oxygen reservoir in a lactate sensor, where oxygen levels are monitored using an independent oxygen biosensor.

FIG. 4 is a plot of current (nA) versus time depicting the stability in sensitivity to lactate of the biosensors in fluctuating oxygen conditions, where oxygen levels are monitored using an independent oxygen biosensor.

### DETAILED DESCRIPTION OF THE DRAWINGS

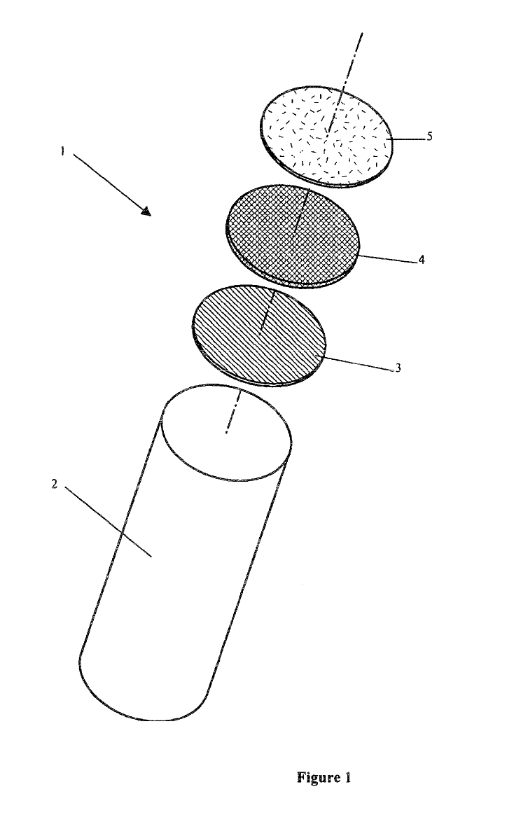

FIG. 1 is a schematic representation of an EBS incorporating an electrode structure of the invention. The electrode 1 comprises a transducer which in the embodiment is a Pt wire 2. On the wire 2 is an interference rejection membrane 3 and an immobilisation matrix 4. Each are represented schematically as discs of material for the purpose of illustration. It will be appreciated that the materials may cover any desired amount of the Pt wire surface. It will be further appreciated that while the various components are illustrated as clearly identifiable layers there will generally be intimate contact and intermixing between the layers. Indeed the top layer 5 of material is a schematic representation of enzymes and/or stabilisers for the purpose of separate identification when indeed they will generally be mixed with one or more other components on the electrode. In particular it will be appreciated that the immobilisation matrix will generally incorporate the enzyme (and optionally stabilisers for the enzyme) and the oxygen reservoir material so that the oxygen reservoir material provides sufficient oxygen to allow the enzymatic process requiring oxygen to continue without any substantial effect on the catalytic process of oxygen fluctuation (and in particular oxygen depletion) in the environment of use.

In the embodiment the interference rejecting membrane 3 comprises oPPD. The immobilisation matrix is constructed of a Nafion™/polystyrene matrix and the layer 5 comprises the desired enzyme and optional stabiliser for the enzyme, though as stated above the desired enzyme and optional stabiliser for the enzyme will generally be held within the immobilisation matrix.

It will be appreciated that an electrode as described above and shown schematically in FIG. 1 can be made in many ways. One method of obtaining such a structure is described in the Experimental Detail below.

### Experimental Details

The sensor can be constructed on any surface that can be charged with an electric voltage. In the course of the present inventors' research Pt wire was used. A primary interference-rejection polymer layer (corresponding to rejecting membrane 3 of FIG. 1) was then electrochemically grown onto the surface of the sensor. This was done by exposing the sensor surface to a 300 mM monomer solution of oxygen-purged o-phenylenediamine in an oxygen deprived environment. A potential of +700 mV (vs SCE) was then applied for a 30 min period. (As the polymer is non-conducting, it has a self-limiting thickness and the application of the potential for any further time period will not affect the polymer thickness or condition.) After this, the sensor is washed with distilled, deionised water and allowed to dry.

The outer styrene-based polymer matrix, into which the enzyme is incorporated, was then applied by a non-electrochemical dip-coating method. The sensor surface was then placed into an initial solution of Styrene: 5% Nafion with a 9:1 ratio. The sensor was then immediately placed into solution of the required oxidase enzyme, immediately thereafter into a 1% PEI (polyethylenimine) solution and finally into a 1% BSA:0.1% Glut (bovine serum albumin:glutaraldehyde) solution. The sensor was then allowed to dry for 5 minutes. After the allotted drying time, the dip-coating of the enzyme, PEI and BSA:Glut was repeated a further nine times allowing 5 min drying interval between each coating. The sensors were then allowed to dry for a sufficient period of time so as to cure the polystyrene polymer matrix. (This time frame can be anything from 1 hour to overnight) After such drying time the full dip-coating procedure, including the Styrene:Nafion coating, was repeated. The sensors were then allowed to dry, (overnight) before being washed with distilled, deionised water. (This resulted in an immobilisation matrix 4 which incorporates the enzyme/stabiliser layer 5 of FIG. 1 so that the materials are intermixed and can be considered to be in a single hybrid matrix). These electrodes were also constructed whereby the Styrene:5% Nafion solution was replaced with a pure styrene monomer solution. This non-oxygen reservoir containing sensor was to allow for comparison purposes.

Remarks

While the enzyme immobilization matrix is used here in conjunction with an o-PPD interference rejection polymer film, it is not necessary to include such a film should the sensor be used in non-vivo situations where interference is not anticipated. Moreover, should the target environment present different interfering species, the primary interference rejecting polymer can easily be amended to suit the situation.

This sensor technology, of which a specific embodiment is described above overcomes these three major issues by providing a seemingly universal surface whereby enzymes can fully function and interact with the target substrates, where all their required conditions are fulfilled with the incorporation of the oxygen reservoirs, while also incorporating a “sieve” system on the surface to ensure that only the H₂O₂ produced by the reaction of the target substrate with the enzyme reaches the charged sensor surface. The resulting sensor is highly sensitive to minute changes, real time, and highly selective to the target substrate. These two factors mean that the sensors can be constructed economically.

Measurements

Two electrodes of the invention were prepared as set out above. The first was configured as a glutamate sensor, the second was configured as a lactate sensor. Both were utilised to take measurements as set out below.

Firstly, the bare platinum electrode was prepared to act as an oxygen reference electrode. This was done by trimming a length of Teflon coated platinum wire to a length of approximately 50 mm and 5 mm of the Teflon was removed from one end to allow for electrical contact. This exposed end of the wire was then soldered into a standard gold contact for rigidity.

These sensors were calibrated at +700 mV vs SCE and in the standard 3 electrode cell in PBS (pH 7.4) under standard laboratory conditions to determine sensitivity to the target substrate.

The sensors were then tested for fluctuation in substrate sensitivity with respect to oxygen fluctuations. For these experiments, the cell was arranged so that one of the working electrodes was the enzyme-based sensor to detect the target substrate with an applied potential of +700 mV vs. SCE, and the other working electrode was a bare Pt disc electrode with an applied potential of −650 mV vs. SCE to detect fluctuations or changes in the concentration of molecular oxygen. The electrodes were allowed to settle in a totally inert environment. A background current level for both electrodes was obtained. While maintaining the inert environment, an aliquot of the target substrate was introduced into the cell solution. From this point, the current values for both working electrodes were continuously recorded for the duration of the experiment. Once a suitable steady-state current value had been recorded, the nitrogen supply was removed from the cell and an air supply was immediately introduced over the cell solution. The experiment was continuously monitored until both electrodes reached a well-established steady-state.

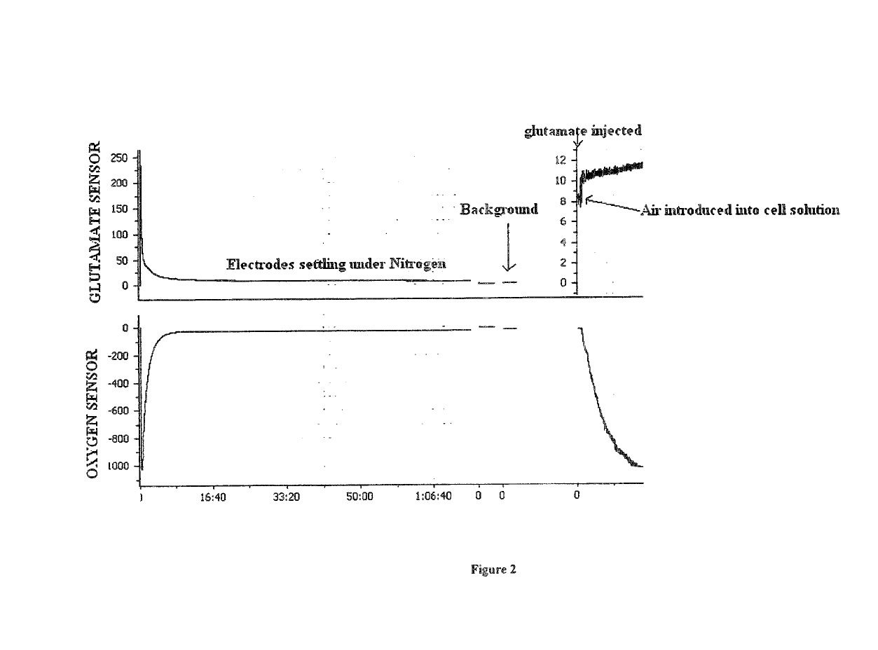

The experimental runs described above generated the data plotted in FIGS. 2 to 4.

FIG. 2 illustrates the difference in sensitivity experienced by the glutamate biosensors while independently monitoring the fluctuations in available oxygen levels with a bare platinum electrode. In the glutamate sensor initial injection of glutamate in the presence of oxygen results in a marginal concentration-dependant change in the current recorded. whereas under similar conditions injection of glucose into the glucose biosensor results in a much more substantial change to the current recorded. However, once within the physiological oxygen concentration region (30-50 μM) and above, there is no significant change with the glutamate signal.

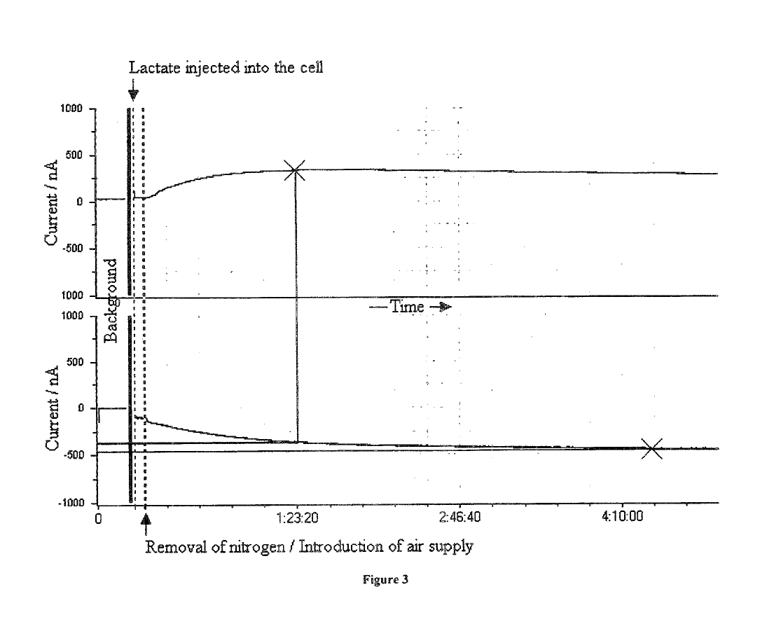

In FIG. 3 a plot of current (nA) versus time in the absence of an oxygen reservoir in a lactate sensor is illustrated. Under an atmosphere of nitrogen lactate injection into the cell produces little or no change in the current recorded relative to the background reaction (as indicated in the Figure). The introduction of an air supply into the cell results in a surge of current, substantiating the dependence of the sensor on molecular oxygen. The curve reaches a plateau at approximately 1:23:20 (shown by the vertical line connecting the two mirror image plots) indicating that the sensor is now saturated with oxygen, and the current at total air equilibration (240 uM O₂) is denoted by the second horizontal line in the lower plot.

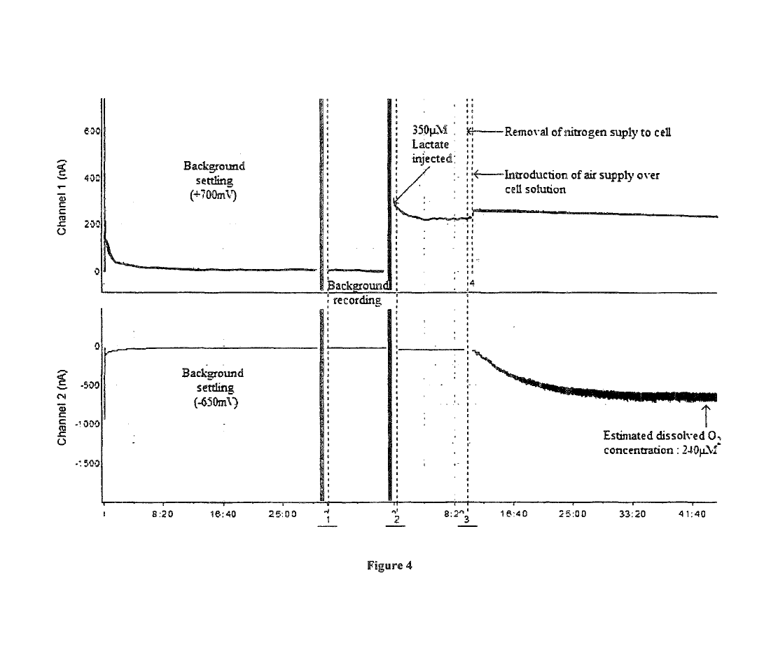

A plot of current (nA) versus time when oxygen reservoirs are present in the polymer of the lactate sensor is shown in FIG. 4. Following lactate injection the electrode reaches full sensitivity to the target substrate almost immediately and increasing the available oxygen concentration through the removal of nitrogen results in no deviation in sensitivity (the slight decline in the sensor signal occurs because the target substrate in the cell is being used up over time and so the sensitivity drops accordingly). The lower plot indicates that at total air equilibration from a nitrogen-saturated solution to where the estimated dissolved O₂ is 240 uM.

### CONCLUSION

FIG. 3 shows the dependency of the biosensors on molecular oxygen. In the absence of oxygen the catalytic cycle cannot be completed, and no change in the current recorded is observed. When the ambient environment is flushed with oxygen there is a rapid change in the current recorded.

FIGS. 2 and 4 show the behaviour of the biosensors where the presence of oxygen reservoirs in the polymer matrix provide the necessary oxygen to complete the catalytic cycle. The electrodes of the invention are thus shown to be substantially insensitive to fluctuations in oxygen levels in the measuring environment and in particular insensitive to depletion in oxygen levels.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

1. An electrode comprising:
(i) a conducting substrate for detecting an electroactive agent;
(ii) a catalytic agent held on the substrate for converting a non-electroactive agent to an electroactive agent by a catalytic process which consumes oxygen; and
(iii) an oxygen reservoir, for releasing oxygen to feed the catalytic process, held within a polymer matrix on the substrate.

2. An electrode according to claim 1 wherein the conducting substrate comprises a non-metallic conductor such as carbon fibres or metallic conductors including those constructed of noble metals such as Pt, Ag, Au, Ru, Rh, Pd, Re, Os, Ir and combinations thereof.

3. (canceled)

4. (canceled)

5. An electrode according to claim 1 wherein the catalytic agent is selected from oxidase materials.

6. An electrode according to claim 3 wherein the catalytic agent is selected from glucose oxidase, lactate oxidase and glutamate oxidase and combinations thereof.

7. An electrode according to claim 1 wherein the oxygen reservoir comprises at least one halogenated polymer such as fluorinated or chlorinated polymers for example, perfluoro, perchloro and perfluorochloro polymers.

8. An electrode according to claim 1 wherein the oxygen reservoir comprises at least one halogenated polymer such as fluorinated or chlorinated polymers for example, perfluoro, perchloro and perfluorochloro polymers.

9. (canceled)

10. (canceled)

11. (canceled)

12. An electrode according to claim 1 wherein the polymer matrix is applied by a non-electrochemical method of application.

13. (canceled)

14. An electrode according to claim 1 wherein the oxygen reservoir is incorporated into the same matrix as the catalytic agent.

15. An electrode according to claim 1 further comprising a cross-linking agent for more securely binding the catalytic agent into the polymer matrix or further comprising a stabiliser for the catalytic agent.

16. (canceled)

17. A method of constructing an electrode comprising:
(i) providing a conducting substrate for detecting an electroactive agent;
(ii) providing on the substrate a catalytic agent for converting a non-electroactive agent to an electroactive agent by a catalytic process which consumes oxygen; and
(iii) providing within a polymer matrix on the substrate, an oxygen reservoir, for releasing, in use, oxygen to feed the catalytic process.

18. A method according to claim 10 wherein the conducting substrate comprises a non-metallic conductor such as carbon fibres or metallic conductors including those constructed of noble metals such as Pt, Ag, Au, Ru, Rh, Pd, Re, Os, Ir and combinations thereof.

19. (canceled)

20. (canceled)

21. An electrode according to claim 10 wherein the catalytic agent is selected from oxidase materials.

22. A method according to claim 12 wherein the catalytic agent is selected from glucose oxidase, lactate oxidase, glutamate oxidase and combinations thereof.

23. A method according to claim 10 wherein the oxygen reservoir is constructed from at least one halogenated polymer such as fluorinated or chlorinated polymers for example, perfluoro, perchloro and perfluorochloro polymers.

24. A method according to claim 10 wherein the oxygen reservoir is constructed from oxygen reservoir material dispersed within a polymeric layer, and optionally within discrete areas of the polymeric layer.

25. (canceled)

26. (canceled)

27. A method according to claim 10 wherein the polymer matrix is applied by a non-electrochemical method of application.

28. A method according to claim 16 wherein the polymer matrix is applied by dip-coating or droplet evaporation.

29. A method according to claim 10 wherein the oxygen reservoir is incorporated into the same matrix as the catalytic agent.

30. A method according to claim 10 further comprising employing a cross-linking agent for more securely binding the catalytic agent into the polymer matrix.

31. A method according to claim 10 further comprising employing a stabiliser for the catalytic agent.
